(19) United States
(12) Patent Application Publication
(10) Pub. No.: US 2024/0225512 A1
(43) Pub. Date: Jul. 11, 2024

(54) STRAIN-ISOLATED SOFT BIOELECTRONICS FOR WEARABLE SENSOR DEVICES

(71) Applicant: GEORGIA TECH RESEARCH CORPORATION, Atlanta, GA (US)

(72) Inventor: Woon-Hong YEO, Atlanta, GA (US)

(21) Appl. No.: 18/563,796

(22) PCT Filed: May 27, 2022
(86) PCT No.: PCT/US2022/031346

§ 371 Date: Nov. 22, 2023

Related U.S. Application Data
(60) Provisional application No. 63/194,109, filed on May 27, 2021.

(51) Int. Cl. A61B 5/28; A61B 5/00; A61B 5/27

(52) U.S. Cl. CPC A61B 5/28; A61B 5/27; A61B 5/681; A61B 2562/0219; A61B 2562/164; A61B 2562/187

(57) ABSTRACT

An exemplary system and method are disclosed for a wearable soft bioelectronic system configured with strain isolators that can isolate its sensor electrode or other sensors in proximity or in contact with the skin from temporary stretching and relative motion of the skin due to gross body movements, e.g., walking. The exemplary system employs hard-soft materials and an isolation structure that facilitates the use of a wearable sensor that can be placed on the surface of the skin and minimize motion artifacts in the acquired signals during physical motion by the wearer. The exemplary system can employ stretchable sensors in combination with the strain isolators.

### RELATED APPLICATION

This PCT application claims priority to, and the benefit of, U.S. Provisional Patent Application No. 63/194,109, filed May 27, 2021, entitled “Strain-Isolated Soft Bioelectronics for Wireless, Continuous, Motion Artifact-Controlled Health Monitoring in Real-Life Activities,” which is incorporated by reference herein in its entirety.

### STATEMENT OF GOVERNMENT INTEREST

This invention was made with government support under grant no. 2024742 awarded by the National Science Foundation. The government has certain rights in the invention.

### BACKGROUND

Portable, long-term, continuous monitoring of biophysical signals acquired via wearable devices is generally desired, for example, for everyday IoT wearable devices such as smart watches as well as in clinical settings, for example, wearable electrocardiograms. Collecting high-quality data remains challenging due to motion artifacts.

A motion artifact (MA) generally includes a temporary change in a measured voltage caused by the movement of the sensor and/or body where the sensor is located. For example, walking can create a downward force on the skin and the sensor device with every step, which can cause a temporary stretching of the skin and relative motion of the skin with the electrode. Together, these two disturbances can change the half-cell potential of the skin as well as the contact impedance with the electrode, respectively. These temporary changes in the measured voltage can have the same amplitude and frequency as other body signals, such as heart contractions, making them often difficult to distinguish from many physiological signals. While software algorithms and signal filtering are commonly used to improve signal quality, they can be computationally expensive, especially for long-term monitoring, and they may still only provide an estimate of the actual biosignal.

There is a benefit to improving biophysical signal acquisition without or with reduced motion artifacts.

### SUMMARY

An exemplary system and method are disclosed for a wearable soft bioelectronic system (also referred to herein as “SIS”) configured with strain isolators that can isolate its sensor electrode or other sensors in proximity or in contact with the skin from temporary stretching and relative motion of the skin due to gross body movements (e.g., walking). The exemplary system employs hard-soft materials and an isolation structure that facilitates the use of a wearable sensor that can be placed on the surface of the skin and minimize motion artifacts in the acquired signals during physical motion by the wearer. The exemplary soft bioelectronic system may also employ stretchable sensors in combination with the strain isolators.

In some embodiments, the wearable soft bioelectronic system and strain-isolated sensor electrode can be used in combination with inertia sensors, e.g., for health monitoring during everyday activities, e.g., a portable ECG device, a health monitoring device. A study was conducted through analytical and computational analysis and dynamic experiments that confirm the utility of the exemplary system in removing or reducing motion artifacts in wearable sensor devices.

In an aspect, a system (e.g., sensor system or device system) is disclosed. The system can include a flexible substrate comprising two or more low-modulus layers including a top low-modulus layer and a bottom low-modulus layer, the flexible substrate having a first side on the top low-modulus layer and a second side on the bottom low-modulus layer, where the second side is configured as a breathable soft membrane configured to directly contact and adhere with a skin region of a person; one or more pads (e.g., an electrode, a stretchable electrode, a set of pads for mounting sensor ICs, or a set of stretchable pads) fixably attached to the second side of the flexible substrate, where the one or more pads include a first pad that attaches to the second side over a first area, where the one or more pads each has a side that is exposed to directly contact a portion of the skin region; and one or more strain-isolating structure fixably attached to the top low-modulus layer, including a first strain-isolating structure, where the first strain-isolating structure is attached to the top low-modulus layer at an area corresponding to the first area of the first pad and is shaped to have an outer dimension that forms a perimeter around the first pad (e.g., where the strain-isolating structure prevent or reduce temporary changes in pad impedance caused by skin strain and pad movement or sliding).

In some embodiments, the first strain-isolating structure has an inner dimension that extends beyond the first area of the first pad.

In some embodiments, the first pad has a first shape, and the first strain-isolating structure has a second shape, where the first shape of the first pad is the same as the second shape of the first strain-isolating structure.

In some embodiments, the first pad has a first shape, and the first strain-isolating structure has a second shape, where the first shape of the first pad is different from the second shape of the first strain-isolating structure.

In some embodiments, the first pad has a shape selected from the group consisting of a square area, a rectangular area, a circular area, or an oval area.

In some embodiments, the first pad has a planar geometric shape.

In some embodiments, the top low-modulus layer includes a thin low-modulus silicone elastomer material having an average thickness less than 1000 um.

In some embodiments, the bottom low-modulus layer includes a low modulus silicone gel material having a modulus value less than 20 kPa.

In some embodiments, the top low-modulus layer includes a first material having a first modulus value, and the bottom low-modulus layer includes a second material having a second modulus value, where the first modulus value of the top low-modulus layer is at least 2 times that of the second modulus value of the bottom low-modulus layer.

In some embodiments, the one or more pads form an array.

In some embodiments, the one or more pads are configured as an open mesh stretchable electrode.

In some embodiments, the open mesh stretchable electrode includes a plurality of stretchable pads (e.g., circular, oval, square, rectangular, or other geometric shapes) each connected together by serpentine or meandering mesh connections.

In some embodiments, the system is configured as a smartwatch configured for at least one of optical measurement, impedance measurement, capacitance measurement, or voltage potential measurement through the stretchable pads.

In some embodiments, the system includes a second stretchable pad that attaches to the second side over a second area, and a second strain-isolating structure attached to the top low-modulus layer at a third area corresponding to the second area of the second stretchable pad and is shaped to have an outer dimension that forms a perimeter around the second stretchable pad.

In some embodiments, one or more pads (e.g., stretchable pads), including the first pad, form a pair of contacts that is fixably attached to the second side of the flexible substrate over a second area, the system further comprising: an active integrated sensor component that couples to the pair of stretchable contacts.

In some embodiments, the active integrated sensor component includes a light-emitting diode or a photodiode.

In some embodiments, the system is configured as an electrocardiographic probe.

In some embodiments, the electrocardiographic probe includes a pair of the flexible pads, a multi-axis accelerator (e.g., 3-axis accelerator), and a multi-axis gyroscope (e.g., 3-axis gyroscope).

In some embodiments, the electrocardiographic probe further includes an optical sensor, a photodiode, a capacitance, and/or a temperature sensor.

In some embodiments, the system includes an active integrated chip assembly mounted on the flexible substrate, where the active integrated chip assembly includes at least one or more active integrated circuits, including a transimpedance amplifier circuit and a digital-to-analog convertor.

In some embodiments, the system includes an encapsulation layer that encapsulates the active integrated chip assembly.

In some embodiments, the active integrated chip circuit further includes a local processing unit or controller (e.g., silicon or IC) configured to provide measured signal data to a device processing unit or controller (e.g., for a smartwatch).

In some embodiments, the device processing unit or controller is configured to employ at least one of electrocardiogram signals, heart rate signals, respiration rate signals, or a combination thereof acquired through an electrode or sensor associated with the one or more pads (e.g., stretchable pads).

### BRIEF DESCRIPTION OF THE DRAWINGS

The skilled person in the art will understand that the drawings described below are for illustration purposes only.

FIG. 1 shows an example wearable soft bioelectronic system device configured with a strain isolated sensor in accordance with an illustrative embodiment.

FIGS. 2A-2J each shows example configurations of the strain isolating structure in the isolated sensor device or system of FIG. 1 in accordance with an illustrative embodiment.

FIGS. 3A-3E each provides examples of strain isolator configurations in accordance with an illustrative embodiment.

FIG. 4 shows a method for fabricating a breathable substrate for use in a strain-isolated sensor device in accordance with an illustrative embodiment.

FIG. 5A shows an example fabrication method for the isolated sensor device of FIG. 1 in accordance with an illustrative embodiment.

FIG. 5B shows an example fabrication method for a stretchable sensor electrode or pad that could be used in a strain-isolated sensor device in accordance with an illustrative embodiment.

FIG. 6 shows an overview of a study that was conducted to develop and evaluate a health monitoring device configured with strain isolating materials and structure in accordance with an illustrative embodiment.

FIG. 7 shows an analytical framework for the strain-isolation physics and computational modeling conducted in the study of FIG. 6.

FIG. 8 shows results from an evaluation of the signal processing and classification performance of the strain-isolated sensor system in the study of FIG. 6.

FIG. 9 shows the results from the comparison study between the strain-isolated sensor system and a commercial sensor system in the study of FIG. 6.

FIG. 10 shows the long-term performance of the strain-isolated sensor system, e.g., to measure physiological signals during real-life activities evaluated in the study of FIG. 6.

FIG. 11 shows detailed implementation of the classification algorithm of FIG. 8.

FIG. 12 shows an experimental setup to measure adhesion strength on a sensor device placed on the forearm of a person.

FIG. 13 shows a skin-electrode impedance experiment that identified strain as a cause of motion artifact for wearable devices.

### DETAILED SPECIFICATION

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entireties and to the same extent as if each reference was individually incorporated by reference.

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. For example, [1] refers to the first reference in the list. All references cited and discussed in this specification are incorporated herein by reference in their entireties and to the same extent as if each reference was individually incorporated by reference.

### Example System

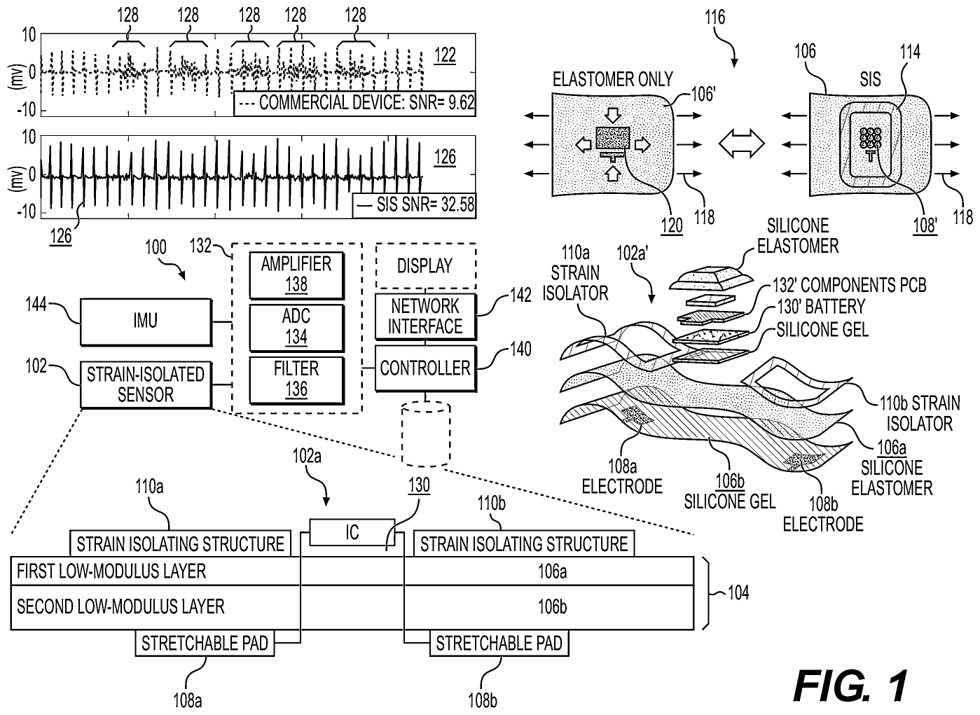

FIG. 1 shows an example wearable soft bioelectronic system device 100 configured with a strain isolated sensor 102 (shown as 102a) according to an illustrative embodiment. The strain isolated sensor 102 includes, or is formed of, a flexible substrate 104 having one or more low-modulus layers 106 (shown including at least a first low-modulus layer 106a, and a second low modulus layer 106b), the flexible substrate 104 is fixably connected to one or more sensor pads 108 (shown as 108a, 108b) and corresponding strain isolators 110 (shown as 110a, 110b) (also referred to as strain isolating structures). In the example shown in FIG. 1, the flexible substrate 104 can be made of a silicone elastomer as a first low-modulus layer and a silicone gel as a second low-modulus layer; the second low-modulus layer has a lower modulus than the first low-modulus layer. The flexible substrate 104 includes a contact portion (e.g., the second low-modulus layer 106b) comprising a breathable soft membrane material configured to adhere to the skin of a person. The one or more sensor pads 108 (shown as 108a, 108b) are preferably formed or attached to the flexible substrate 104 to serve as either (i) an electrode of the sensor or (ii) as a connecting pad for an integrated sensor that can contact or be in proximity to the skin. Non-limiting examples of integrated sensors that can be included mounted to the pad 108 include an optical sensor, a photodiode, a capacitance, a temperature sensor, a combination thereof, or other sensors described herein.

The position of the strain isolator(s) 110 can correspond to the position of a respective sensor pad 108. The strain isolator 110 can be shaped to have an outer dimension 112 that forms a perimeter around the sensor pad (e.g., 108a). By forming a perimeter 114 around the sensor pad (e.g., 108a), the strain isolator 110 can prevent or reduce the temporary changes in pad impedance caused by skin strain and pad movement or sliding. An illustration of the concept is shown in diagram 116. In diagram 116, a sensor electrode is shown formed on an elastomer without the strain isolator 114. As tension 118 (e.g., uniaxial strain) from temporary stretching of the skin or relative motion of the skin with the electrode is applied to the elastomer 106′, a resulting deformation is also observed on the sensor 120. In contrast, when tension 118 is applied to the strain isolated sensor 108 (shown as 108′), there is no corresponding tension or stretching observed at the strain isolated sensor 108′.

Plot 122 shows an example biophysical signal 124′ acquired from a commercial electrocardiographic electrode. Plot 126 shows a biophysical signal 124 acquired from an electrocardiographic electrode configured with a strain isolator sensor as described herein. It can be observed in plot 126 that the same signal has a higher signal-to-noise ratio (SNR) (32.58 dB as compared to 9.62 dB), and that the artifacts 128 produced by body motions in plot 122 are not present in plot 126 to provide a higher quality signal and higher SNR.

The low-modulus layers 106 of the flexible substrate 104 can form a unitary structure that can flex or bend in a pre-defined orientation. In some embodiments, the flexible substrate 104 may have a single low-modulus layer having a gradient or varying modulus among its top regions and its bottom regions. In other embodiments, the flexible substrate 104 may be formed of multiple layers, each having a different modulus property.

In an example shown in FIG. 1, the strain isolated sensors 102 (shown as 102a′) may be formed of a thin low-modulus silicone elastomer material (e.g., having an average thickness less than 1000 um) as a first low-modulus layer 106a, and the second low-modulus layer 106b is formed of a low modulus silicone gel material having a modulus value less than 20 kPa. Indeed, the modulus value of the first low-modulus layer 106a is at least two times that of the second low-modulus layer 106b.

In some embodiments, the one or more sensor pads 108 may form an array. The array can be individually located within the perimeter of a strain isolator 114, or the array having the multiple sensor pads may be located within the strain isolator 114.

In some embodiments, the sensor pads 108 are configured as an open mesh stretchable electrode or stretchable pad. The open mesh stretchable electrode or pad provides additional tension or strain relief for the pad structure due to the tension 118 that is applied from the skin and sensor interaction.

### Example Isolated Sensor System

The isolated sensors 102 may be configured as an isolated sensor device comprising IC components. In the example of FIG. 1, the isolated sensor 102a is configured with flexible printed-circuit layer 130 (also shown as “PCB” 130′) to which a front-end acquisition circuitries or components 132 (shown as “components” 132′), such amplifiers (e.g., transimpedance amplifiers), filters, and/or analog-to-digital converters (ADCs), can be mounted. In the example, the isolated sensor 102a includes an analog-to-digital converter 134, filter 136, and amplifier 138.

The isolated sensor device (e.g., 102a′) can be coupled to an external sensor system comprising a controller 140, a network interface 142 (e.g., wireless network interface), and additional sensors 144 (e.g., an inertia measurement unit (IMU) 144 comprising one or more inertia sensors). The controller 140, network interface 142, and/or additional sensors 144 may be mounted on the flexible printed-circuit layer 130 along with the front-end acquisition circuitries or components 132. Non-limiting examples of additional sensors that may be included in the sensor system include temperature, magnetic-based sensor, and acoustic sensors.

In some embodiments, the isolated sensor device (e.g., 102a′) can be fabricated as an integrated sensor system comprising the above-noted components. In the example shown in FIG. 1, the isolated sensor device as a sensor system can include the controller 140, network interface 142, and/or additional sensors 144 as part of components 132′.

### Example Strain Isolating Structure

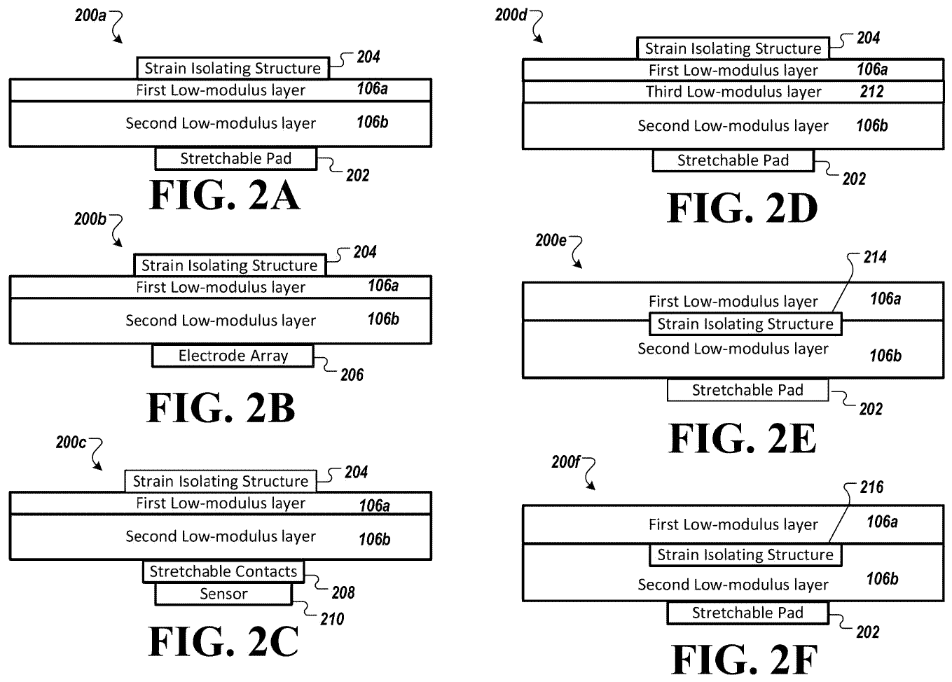

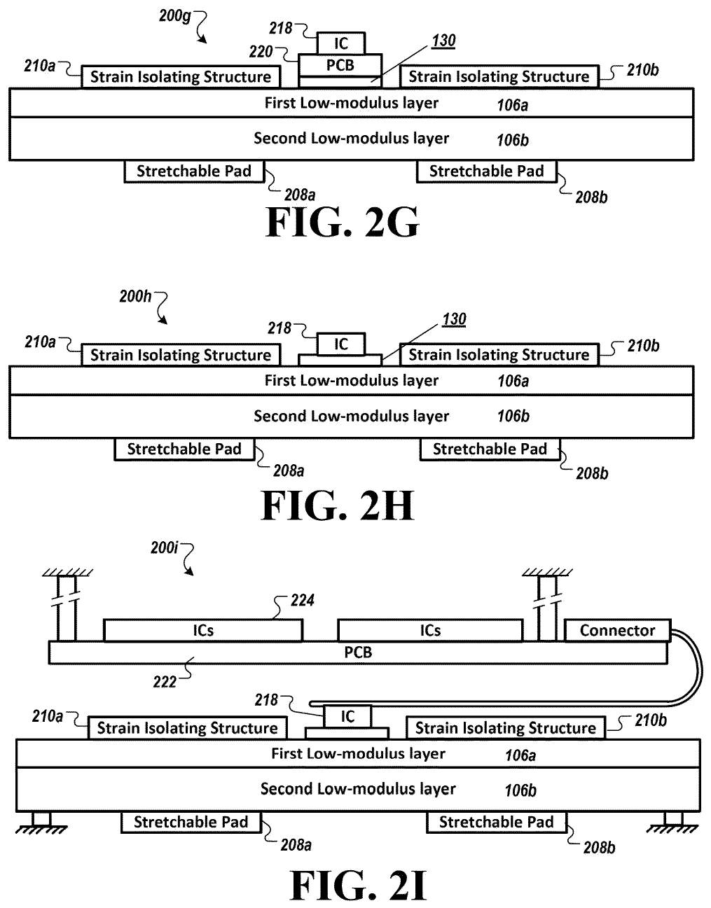

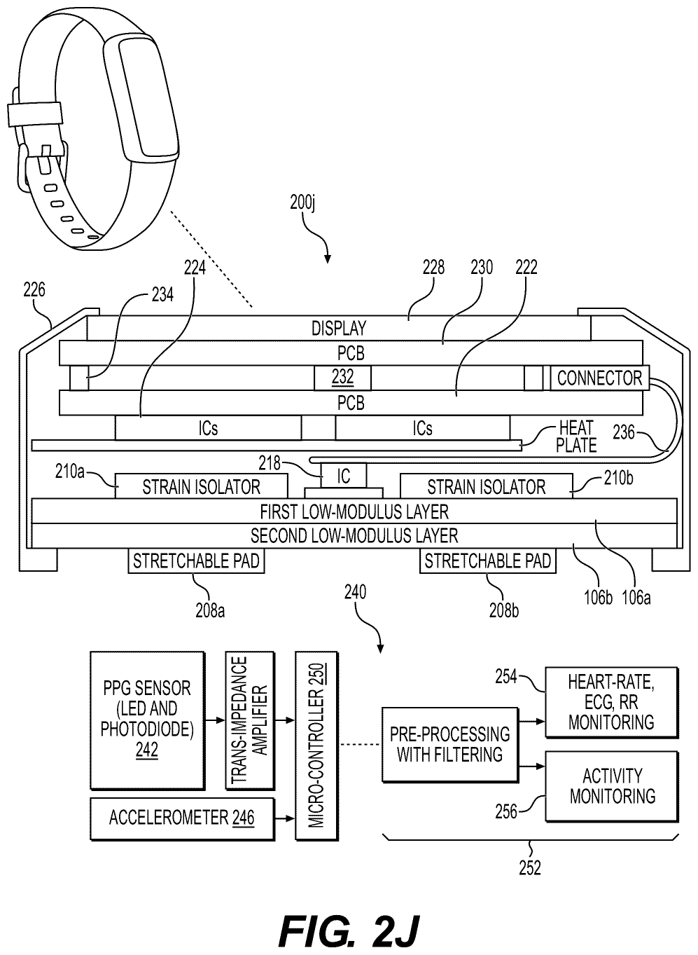

FIGS. 2A-2J each shows example configurations of the strain isolating structure in the isolated sensor device or system of FIG. 1 in accordance with an illustrative embodiment.

Specifically, FIG. 2A shows an example strain isolated sensor 102 (shown as 200a) comprising the first low-modulus layer 106a and the second low modulus layer 106b that has a sensor pad 108 (shown as 202). The strain isolating structure 110 (shown as 204) is located on the top surface of the first low modulus layer 106a.

FIG. 2B shows another example strain isolated sensor 102 (shown as 200b) comprising the first low-modulus layer 106a and the second low modulus layer 106b and strain isolated structure of FIG. 2A. The second low modulus layer 106b includes an electrode array 108 (shown as 206).

FIG. 2C shows another example strain isolated sensor 102 (shown as 200c) comprising the first low-modulus layer 106a and the second low modulus layer 106b and strain isolated structure of FIG. 2A. The second low modulus layer 106b includes an electrode array 108 (shown as 208) that has coupled to it a sensor (e.g., an active integrated sensor component). Non-limiting examples of an active integrated sensor component include a light-emitting diode and a photodiode, a capacitance, and/or a temperature sensor.

FIG. 2D shows another example strain isolated sensor 102 (shown as 200d) comprising the first low-modulus layer 106a and the second low modulus layer 106b and strain isolated structure of FIG. 2A. The flexible substrate further includes a third low-modulus layer 212. The second low modulus layer 106b includes an electrode array 108 (shown as 202). Indeed, the flexible substrate can further include additional layers.

FIGS. 2E and 2F each shows another example strain isolated sensor 102 (shown as 200e and 200f, respectively) comprising the first low-modulus layer 106a and the second low modulus layer 106b and strain isolated structure of FIG. 2A. Here, the strain isolating structure 110 (shown as 214) is located within the flexible substrate. In FIG. 2E, the strain isolating structure 110 (shown as 214) is located in a part of the first low-modulus layer 106a and a part in the second low modulus layer 106b. In FIG. 2F, the strain isolating structure 110 (shown as 216) is located in a part of the second low modulus layer 106b (e.g., recessed in the second-low modulus layer).

FIG. 2D shows another example strain isolated sensor 102 (shown as 200d) comprising the first low-modulus layer 106a and the second low modulus layer 106b and strain isolated structure of FIG. 2A. The flexible substrate further includes a third low-modulus layer 212. The second low modulus layer 106b includes an electrode array 108 (shown as 202). Indeed, the flexible substrate can further include additional layers.

With reference to FIGS. 2G and 2H, another set of example strain-isolated sensors 102 (shown as 200g and 200h, respectively) is shown that each includes the components illustrated in the device 102a of FIG. 1, but additionally with integrated circuits (“IC”) 218. In FIG. 2G, the ICs are mounted to a printed circuit board 220 (flexible or traditional) that is then mounted to the sensor 200g. In the example shown in FIG. 2G, the PCB 220 is mounted to the sensor device 200g through a low-modulus elastomer layer 130. In FIG. 2H, the ICs are mounted to low-modulus elastomer layer 130 directly, e.g., via adhesives. The ICs may be connected through printable wiring, e.g., via conductive paint. As described in relation to FIG. 1, the ICs (silicon or packaged) 218 can include a controller 140, network interface 142, and/or additional sensors 144 as part of components 132′.

FIG. 2I shows another example strain-isolated sensor 102 (shown as 200i) that includes the components illustrated in the device 102a of FIG. 2H, but additionally with the integrated circuits (“IC”) 218 connected to another PCB circuit 222. The PCB circuit 222 includes additional IC components 224 (e.g., silicon or IC) that may be configured to provide measured signal data from the strain-isolated sensor device 102a to a device processing unit or controller (e.g., for a smartwatch or other wearable device). The IC 224 can be a device processing unit or controller configured to employ at least one of electrocardiogram signals, heart rate signals, respiration rate signals, or a combination thereof in an analysis, local or remote storage, or for display, e.g., via a display integrated to the PCB 222.

The processing unit may be a standard programmable processor that performs arithmetic and logic operations necessary for operation of the computing device. Multiple processors may be employed. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

It should be appreciated that the logical operations described above can be implemented (1) as a sequence of computer implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

Additionally, some or all of the components illustrated in and described with reference to FIGS. 1-2I can be encapsulated in an encapsulation layer (not shown).

### Example Smartwatch with Strain-Isolated Sensor

With reference to FIG. 2J, embodiments of the present disclosure (e.g., embodiments described with reference to FIGS. 1A-2I) can be packaged so that some or all of the components are inside a housing or supported by the housing. In the example embodiment 200j illustrated in FIG. 2J, the components illustrated and described with reference to FIG. 2I are positioned in a housing 226. The housing 226 can include, or be adapted to connect to, a strap (e.g., a wristband).

The housing 226 can also include one or more display devices 228 (e.g., digital screens) mounted on a PCB 230. The display PCB 230 can be connected to the PCB 222 through connector 232 and spacers 234. The PCB 222 can connect to the strain-isolated sensor device via IC components 218 through a flexible cable 236. In the example shown in FIG. 2J, the strain-isolated sensor device and system is configured as a wearable, flexible, hybrid electronic system (WFHE).

The smartwatch can be configured to perform measurements, including optical measurement, impedance measurement, capacitance measurement, or voltage potential measurement through the stretchable pads. In the example shown in FIG. 2J, diagram 240 shows the operation of a wearable device (e.g., 200j) configured with a strain-isolated sensor (e.g., 102). Per diagram 240, a PPG sensor (e.g., LED and photodiode) or electrode (shown as 242), either manufactured with the strain-isolated sensor, can acquire biophysical signals along with accelerometer signals (from accelerometer 244) that are provided to a local controller 246 (shown as “Micro controller” 246). The local controller 246 is executing processing operation 252 to preprocess the acquired data and to determine and display the heart rate, ECG, and/or respiration rate measurements 254, and activity monitoring 256. The heart rate measurement 254 may be determined using acquired biophysical signals that are not impacted by motion artifacts. Various activity monitoring can be used, including the one described herein.

Indeed, the strain-isolated sensor can use a breathable soft membrane's natural adhesion to offer a skin-friendly, comfortable, continuous recording of ECG, HR, and RR on the skin and real-time classification of various activities, e.g., via a machine-learning algorithm.

In alternative embodiments, the strain-isolated sensor device and system can be configured as a wrist-worn skin-conformal, soft material-enabled bioelectronic system. Example description is provided in Shinjae Kwon et al., “Skin-conformal, soft material-enabled bioelectronic system with minimized motion artifacts for reliable health and performance monitoring of athletes,” Biosensors and Bioelectronics 151 (2020), which is incorporated by reference in its entirety.

### Examples of Strain Isolator Configurations

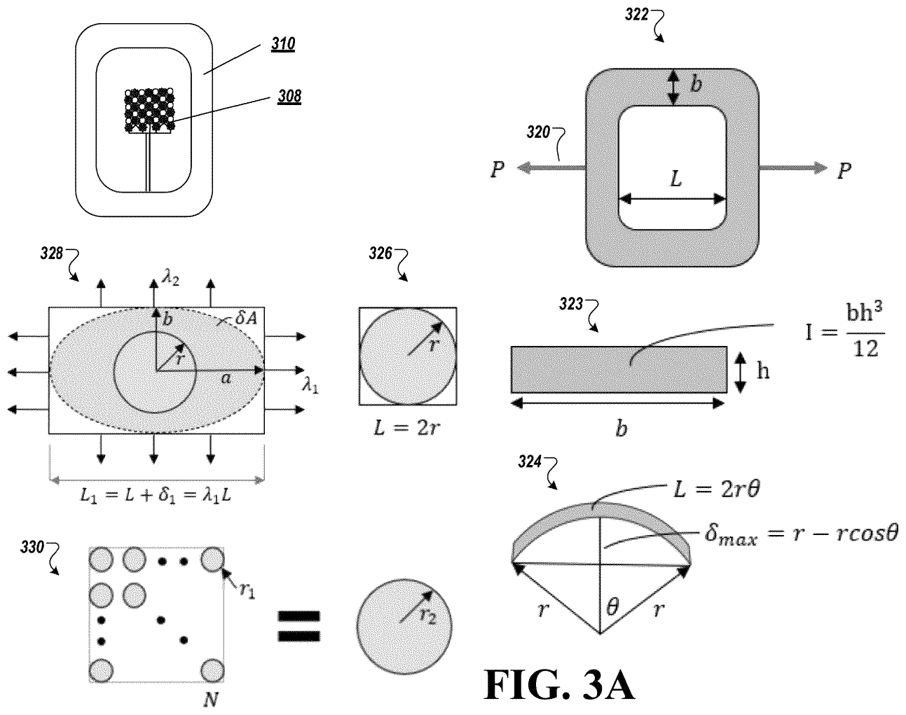

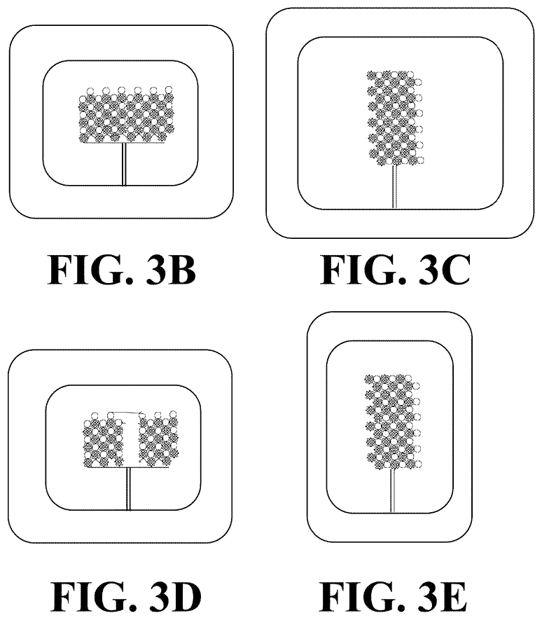

FIGS. 3A-3E provides examples of strain isolator configurations. FIG. 3A shows an example strain isolator 110 (shown as 310) for a given sensor pad 108 (shown as 308) and its associated design. The dimensions and thickness of the strain isolator structure can be defined using the geometry of the electrodes and the associated materials.

With reference to FIGS. 3B-3E, embodiments of the strain-isolated structures can include different configurations with the sensor pad. Indeed, the strain-isolated structure may be formed with different orientations, in different shapes, and using different proportions. In FIG. 3D, multiple sensor pads, electrodes, sensors, or an array thereof, may be surrounded by the strain-isolated structure.

In some embodiments, the first strain-isolating structure has an inner dimension that extends beyond the first area of the first pad.

In some embodiments, the first pad has a first shape and the first strain-isolating structure has a second shape, wherein the first shape of the first stretchable pad is the same as the second shape of the first strain-isolating structure.

In some embodiments, the first pad has a first shape and the first strain-isolating structure has a second shape, wherein the first shape of the first pad is different from the second shape of the first strain-isolating structure.

In some embodiments, the first pad has a shape selected from the group consisting of a square area, a rectangular area, a circular area, or an oval area.

### An Example Configuration for the Strain-Isolated Structure

The strain-isolated structure may be designed based on thickness, materials, and the Young's modulus of the device. An example of the design is presented below.

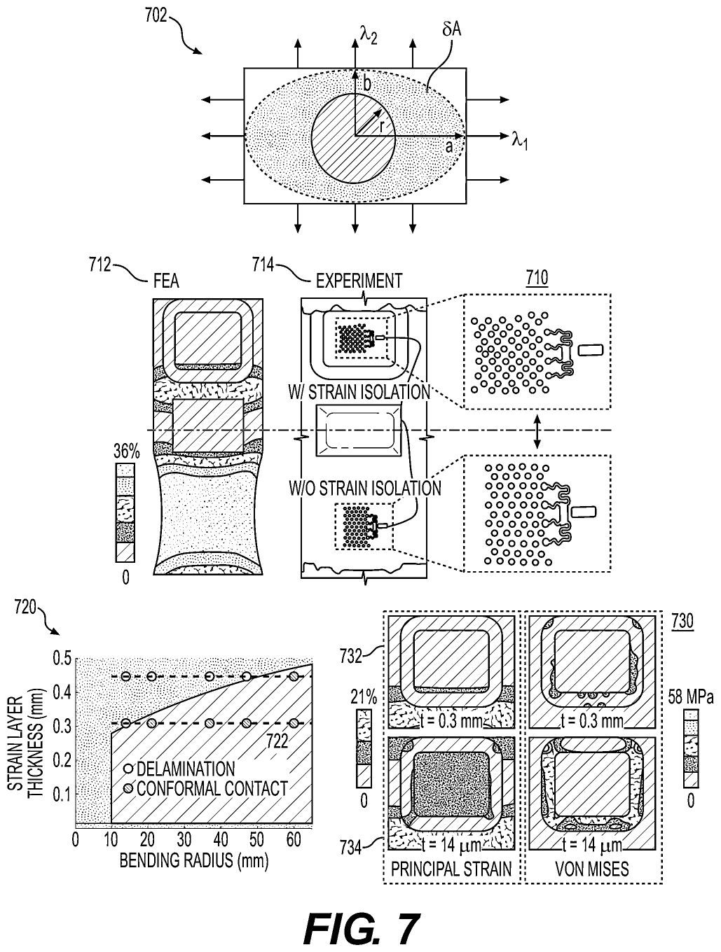

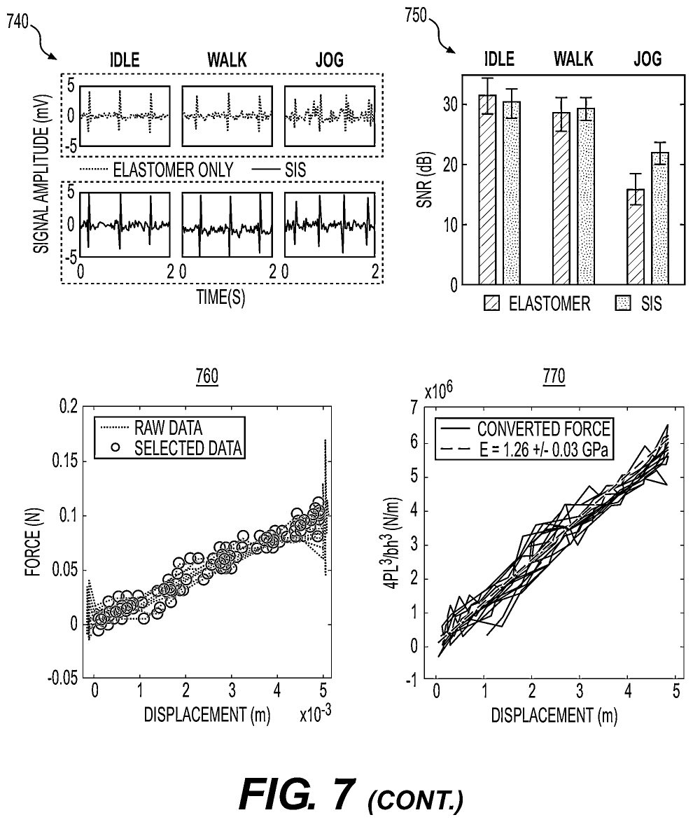

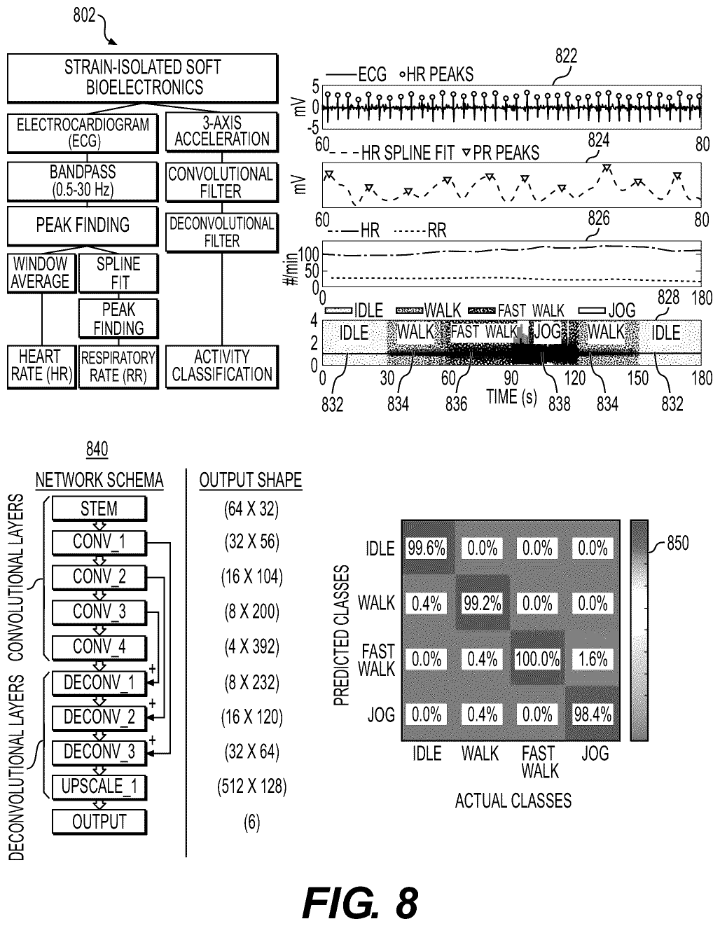

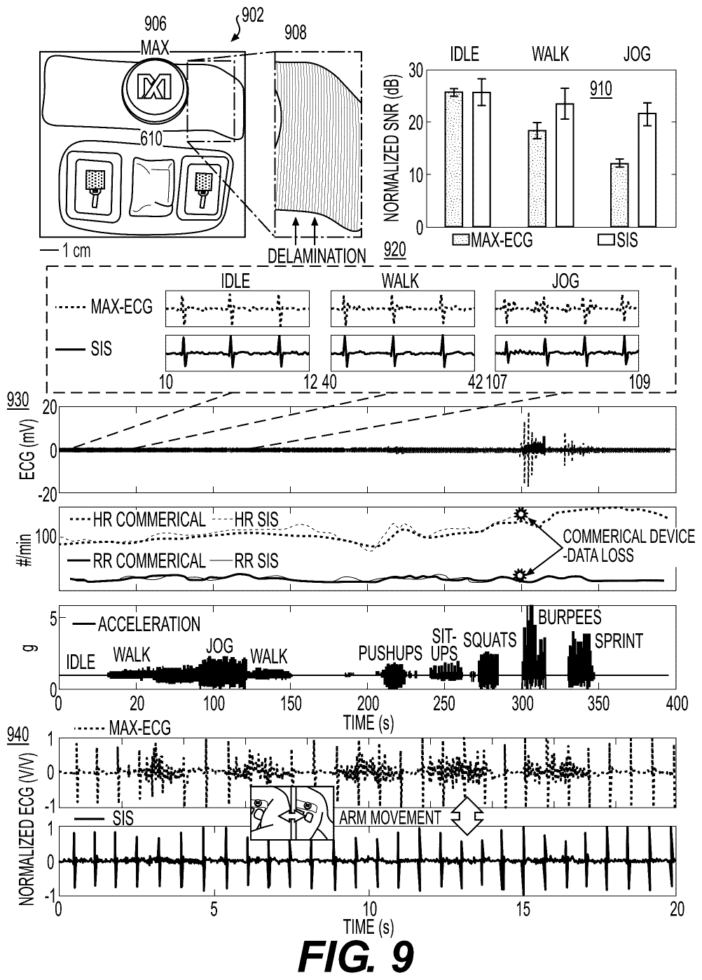

Material characterization. For a sheet modeled as a cantilever beam fixed at one end, the deflection from a force at the tip is calculated per Equation 1:

$$δ = \frac{{PL}^{3}}{3 EI} \qquad ( Eq . 1 )$$

where δ is the deflection at the free end, P is the force (320), L is the beam length, E is Young's modulus, and I is the moment of inertia of a rectangular beam (323), where I=bh³/12. From this equation, the modulus can be calculated per Equation 2:

$$E = ( \frac{P}{δ} ) \frac{4 {L}^{3}}{{bh}^{3}} \qquad ( Eq . 2 )$$

where (P/δ) is the measured value from the bending test and is determined by a linear least-squares fit of the data. An example of the bending test is described in relation to FIG. 7, plots 760 and 770. An example modulus E is 1.22 GPa.

Strain layer optimization. Using the calculated modulus E, and principal mechanics equations, the minimum, and maximum thickness can be calculated to minimize in-plane-strain while allowing for expected out-of-plane bending to maintain conformal contact with the skin. First, the elongation value of the middle section 322 can be calculated for the sheet modeled as a prismatic beam, per Equation 3:

$$δ = \frac{PL}{EA} \qquad ( Eq . 3 )$$

where A is the cross-sectional area for a rectangular beam, and L is the inner dimension of the strain isolated structure. The two edge portions can be modeled as a simply supported beam with point load and maximum deflection in the middle given by Equation 4:

$$δ = \frac{{PL}^{3}}{48 EI} . \qquad ( Eq . 4 )$$

Together, the elongation in the middle section and bending of the edges add up to the total strain inside the perimeter of the strain isolated structure. Combining Equations 2-4 and solving for h results in Equation 5:

$${h}_{min} = \frac{PL}{2 Eb {δ}_{allow}} ( 1 + \frac{{L}^{2}}{12 {b}^{2}} ) . \qquad ( Eq . 5 )$$

where hₘᵢₙ is the minimum sheet thickness required to keep the elongation below δ_{allow}. Example values of P=1 N, L=21 mm, E=1.22 GPa, w=5.5 mm, and δallow=0.64 mm based on 2% strain of the inner dimension L would result in hₘᵢₙ=14.2 μm.

Next, the bending of the sheet and adhesion forces (324) can be modeled as a supported beam with distributed load, given by Equation 6:

$${δ}_{max} = \frac{5 {qL}^{4}}{384 EI} \qquad ( Eq . 6 )$$

where δₘₐₓ is the deflection in the center of the beam, q is the distributed adhesion force per length, L is the beam length, E is the Young's Modulus, and I is the moment of inertia for a rectangular beam. To maintain conformal contact with the skin, the adhesion force should be greater than the beam stiffness for a given deflection. The deflection (per 324) can be determined through the geometry and a radius of curvature related by Equation 7.

$$L = 2 r θ , δ max = r ( 1 - cos θ ) \qquad ( Eq . 7 )$$

Substituting I, δmax, and solving for h results in Equation 8.

$${h}_{max} = \sqrt[3]{\frac{5 {qL}^{4}}{32 Ebr [ 1 - cos ( L / 2 r ) ]}} \qquad ( Eq . 8 )$$

where hₘₐₓ is the maximum sheet thickness capable of maintaining conformal contact with the skin when bent around a radius of curvature r. Example values of q=18.32 N/m based on adhesion testing with silbione, L=32 mm, E=1.22 GPa, b=11 mm, and r=15 mm would result in hₘₐₓ=0.31 mm.

Skin strain modeling. A circular electrode and skin are in an unstrained steady-state configuration (326), and the skin under biaxial strain (328) with stretch ratios λ₁, λ₂. The area of skin in contact with the electrode remains the same. When the skin experiences strain, the electrode moves with the skin but remains relatively unstrained. This loading scenario causes a portion of the skin that is initially in contact with the electrode to slide out of contact with the electrode. If the skin strain is held constant, then a new steady-state can be reached at the current skin/electrode interface. One source of MA is caused by the impedance change that occurs when the skin moves relative to the electrode. The sensitivity of the skin/electrode interface was quantified by the change in the area in contact at the skin/electrode interface. The stretch ratio can be defined as:

$${λ}_{i} = \frac{L + {δ}_{i}}{L} = 1 + {ε}_{i} i = 1 , 2 \qquad ( Eq . 9 )$$

where L is the original length, δᵢ is the elongation, and εᵢ is the strain. Equation 9 can be rearranged to describe the new length per Equation 10:

$${λ}_{i} L = L + {δ}_{i} i = 1 , 2 \qquad ( Eq . 10 )$$

From diagram of the biaxial strain 328, it can be shown that the stretch ratios also define the new half lengths per Equation 11:

$$a = {λ}_{1} r , b = {λ}_{2} r \qquad ( Eq . 11 )$$

The change in the area can be calculated using the area of an ellipse minus the area of the circular electrode per Equation 12:

$$δ A = π ab - π {r}^{2} \qquad ( Eq . 12 )$$

Substituting a, b results in Equation 13:

$$δ A = π {r}^{2} ( {λ}_{1} {λ}_{2} - 1 ) \qquad ( Eq . 13 )$$

Mesh electrodes (330) can be modeled as an N numbered array of small electrodes with a radius r₁, having a total area equal to a single electrode with a radius r₂, the relationship given by Equation 14:

$$A = π {Nr}_{1}^{2} = π {r}_{2}^{2} \qquad ( Eq . 14 )$$

Therefore, the total change in area is shown below to be directly proportional to the strain, regardless of individual radius, for a given total electrode area by Equation 15.

$$δ A = A ( {λ}_{1} {λ}_{2} - 1 ) = π {Nr}_{1}^{2} ( {λ}_{1} {λ}_{2} - 1 ) = π {r}_{2}^{2} ( {λ}_{1} {λ}_{2} - 1 ) \qquad ( Eq . 15 )$$

### Example Breathable Substrate

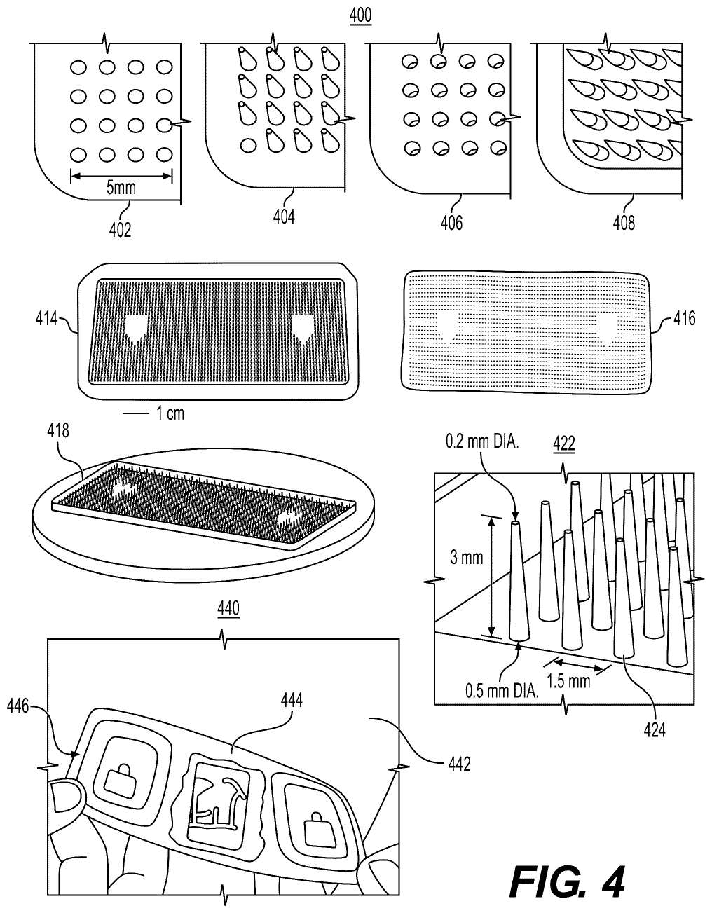

FIG. 4 shows a method 400 for fabricating a breathable substrate for use in a strain-isolated sensor device in accordance with an illustrative embodiment. Method 400 can be used to fabricate an epoxy mold with a needle array. At step 402, holes are laser cut into an acrylic sheet. At step 404, the pattern is copied into flexible DragonSkin needles. At step 406, flexible DragonSkin holes are formed by a copying process. At step 408, a cast final mold of epoxy needle array is created.

FIG. 4 also illustrates a full epoxy needle array 414 and a breathable layer 416 after curing and removal. Embodiments can include a smooth surface to mount the electrodes. An illustration of a CAD model 418 of the epoxy mold is also shown in FIG. 4, with needle and spacing dimensions. An illustration of section 422 of the epoxy mold (e.g., the epoxy mold of the CAD model 418 shown in FIG. 4) includes non-limiting examples of the dimensions and spacing of the needles 424.

The method of fabricating a breathable substrate described in FIG. 4 can be used to form a substate that minimizes skin irritation during and after use. An illustration 440 in FIG. 4 shows the skin 442 where an example device 444 has been removed, and an arrow 446 illustrates the path of the device 444 being peeled away from the skin 442. The skin 442 shows minimal irritation, indicating that the device is breathable. Indeed, the example device can include enhanced breathability compared to devices fabricated using other methods and can be suitable for consecutive data recording during various daily activities. Overall, the example embodiment of an SIS shows a unique performance in strain reduction and real-world applicable continuous recording of health data compared to the existing wearable ECG monitoring systems.

### Example Fabrication Method of Isolated Sensor Device

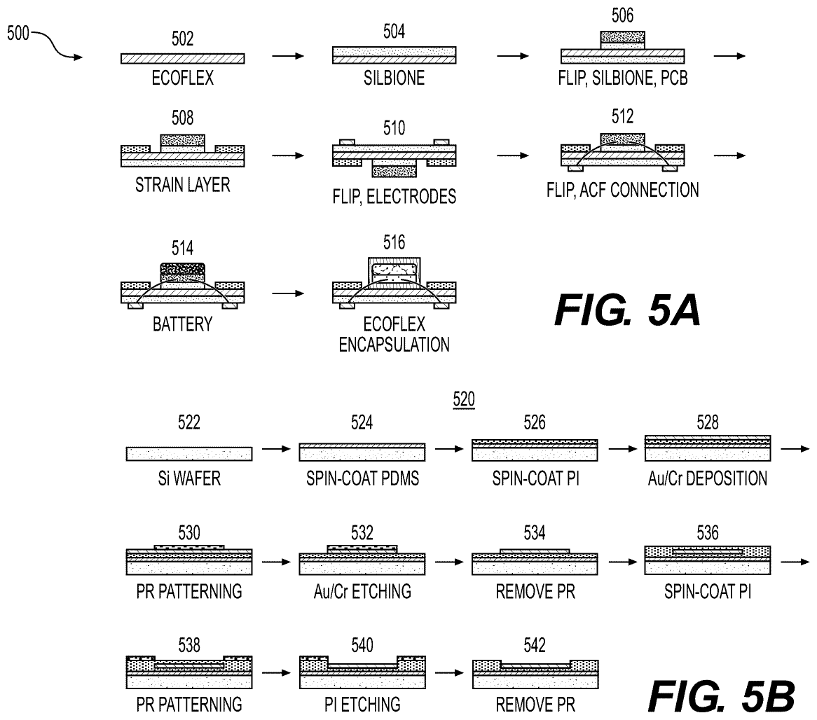

FIG. 5A illustrates an example fabrication method 500 for the isolated sensor device of FIG. 1 in accordance with an illustrative embodiment. In the example shown in FIG. 5A, Method 500 includes preparing (502) an elastomer (e.g., 8 g of Ecoflex 00-30 (Smooth-On)), pouring the elastomer into a container (e.g., a polystyrene petri dish to create a 500 μm thick elastomer membrane), and curing it (e.g., curing Ecoflex at room temperature for 5 hours). Method 500 then includes preparing (504) an elastomer gel (e.g., 8 g of silbione gel A-4717 (Factor II, Inc.)), pouring it over the Ecoflex, curing it (e.g., at room temperature for 24 hours), and cutting to size. Method 500 may then include fabricating (506) a PCB layer. The elastomer can be flipped onto a clean surface with Ecoflex facing up, and a circuit can be attached using a thin film of silbione gel A-4717.

Method 500 may then include attaching (508) a strain isolating layer (e.g., using thin encapsulating layer of Ecoflex without attaching material directly over the electrodes). Method 500 may then include placing (510) the electrodes, e.g., by flipping the elastomer onto a surface (e.g., having a cutout at the circuit to lay flat with the silbione layer facing up). The surface can be cleaned with IPA. Method 500 may then include attaching circuit ICs (e.g., using a thin film of silbione gel A-4717).

Method 500 may then include transferring the flexible electrode to silbione (e.g., by using water-soluble tape and removing the water-soluble table with de-ionized water).

Method 500 may then include connecting the flexible electrodes to the circuit IC using a flexible conductive film (ACF) (e.g., fast-drying silver paint (Ted Pella, Inc.)). Method 500 then includes attaching (514) a battery. Method 500 then includes encapsulating (516) the exposed portions of ACF connections(e.g., with Ecoflex).

### Example Fabrication Method of Stretchable Sensor Electrode or Pad

FIG. 5B shows an example fabrication method 520 for a stretchable sensor electrode or pad that could be used in a strain-isolated sensor device in accordance with an illustrative embodiment.

In the example of FIG. 5B, Method 520 includes forming (or providing) (522) a Si wafer. Method 520 then includes spin coating (524) the wafer with PDMS. Method 520 may then include spin coating (526) polyimide onto the PDMS. Method 520 may then include depositing (528) a layer of gold and a layer of chrome. Method 520 then includes performing (530) PR patterning. Method 520 may then include performing (532) gold/chrome etching. Method 520 may then include removing the PR and spin coating (566) polyimide over the exposed circuit. Method 520 may then include performing (538) PR patterning 568 and then etching (540) the exposed portion to the circuit. Method 520 may then include removing (542) the photoresist.

### Experimental Results and Examples

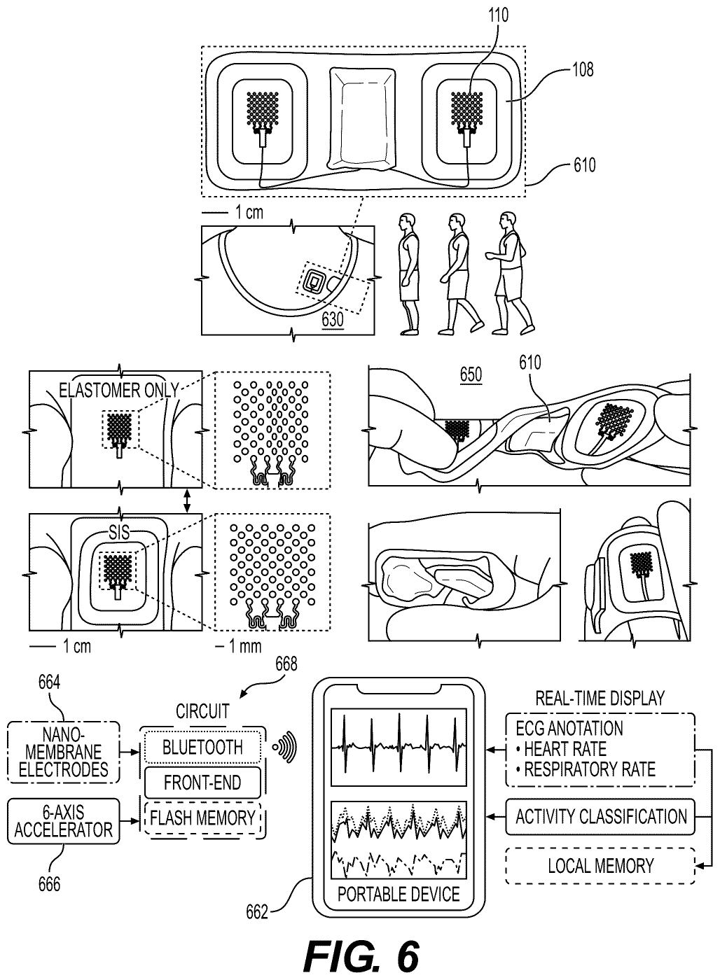

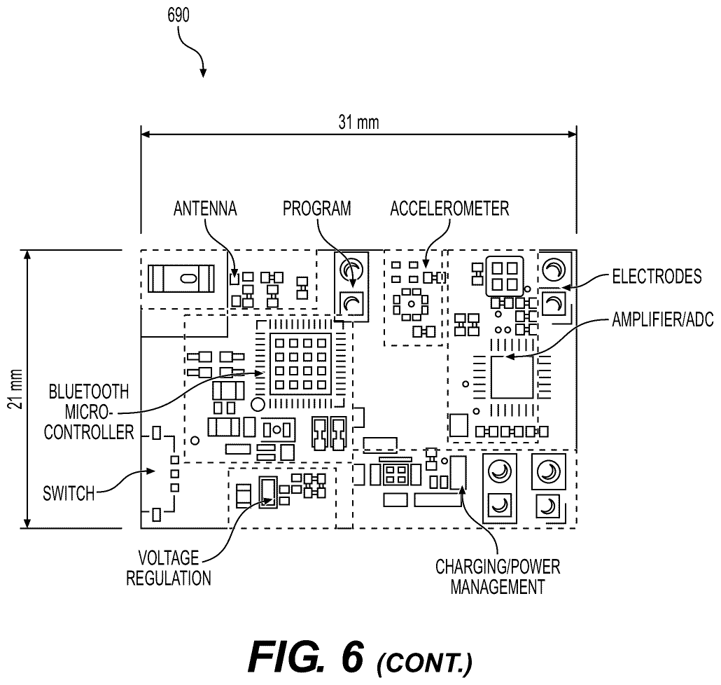

FIG. 6 shows a study that was conducted to develop and evaluate a health monitoring device (e.g., 100) configured with strain isolating materials and structure.

The study developed a new class of strain-isolation physics, hard-soft material integration, and integrated system packaging, enabling an integrated, wireless, long-term usable health monitor. The study, through analytical, computational, and experimental evaluation, observed that the developed wireless, wearable ECG electronic system (e.g., 100) could physically restrict excessive motion artifacts (MA) during real-life, continuous daily activities without losing data. The studied strain-isolated soft bioelectronics (SIS) used a breathable soft membrane to provide natural adhesion that can offer a skin-friendly, comfortable, continuous recording of ECG, heart rate (HR), and respiration rate (RR) on the skin and real-time classification of various activities via a machine-learning algorithm.

The study evaluated, experimentally, the strain-isolated soft bioelectronics with respect to two physiological signal monitoring commercial wireless devices. The study observed exceptional performance of the SIS in maintaining conformal skin contact, enhanced comfort, and high-quality data recording with negligible MA effect. The SIS is simultaneously tested alongside two commercially available wireless devices to show the MA reduction during various physical activities in daily life. Finally, the device is worn by multiple participants for over eight hours, all performing various daily activities ranging from deskwork to exercise.

In FIG. 6, a summary of the design overview is provided of an SIS, structure layouts, strain-isolation mechanics, and the device functions employed in the study. The all-in-one, soft, imperceptible system (shown as 100a in FIG. 6A) can have an exceptionally small form factor that can adhere securely and discretely to the chest area for continuous health and motion monitoring throughout various daily activities.

Initial Skin-Electrode Impedance Analysis. First, a systematic experimental study was conducted that measured skin-electrode impedance to identify the sources of impedance change for a wearable device. FIG. 13 shows a testing setup for disturbance of wires 1330, pressure directly on the electrode 1332, and pressure to the skin 1334 surrounding electrodes. Plot 1340 shows the percentage of impedance changes for each category of disturbance, where the percentage is referenced to the initial value. Plot 1350 shows a plot of the measured impedance vs. time for gel electrodes, and Plot 1360 shows the measured impedance changes vs. time for dry electrodes 1360. Impedance testing was conducted at 100 kHz.

Among three candidates (sensor connecting wires, skin-mounted electrode, and neighboring skin), it was observed that the measured impedance experienced the most significant disturbance from the applied strain to the electrode. The strain isolation sensor was developed in part to address this finding.

Strain Isolation Device. The study developed a pair of strain-isolators to reduce the applied strain and positioned the strain isolator above each electrode. The study also developed a pair of nanomembrane mesh electrodes to make direct contact with the skin for measuring non-invasive physiological signals, such as ECG, HR, and RR. The study designed the open-mesh, stretchable electrode to endure excessive tensile strain up to at least 100% without failure. The study also developed the health monitoring device with a bottom layer having an extremely low modulus silicone gel (e.g., E=5 kPa) to provide excellent adhesive properties to bond the device to the skin in which the top layer has a low modulus silicone elastomer (e.g., E=68.9 kPa) to provide a durable platform to mount the circuit and makes the device more comfortable to handle, and prevents unwanted sticking to clothes. In FIG. 6A, the studied device is shown as 610.

The studied device 610 included a miniaturized PCB with a rechargeable lithium-ion battery (3.7 V, 110 mAh) in which the PCB is encapsulated with a silicone elastomer. The studied device was configured to perform recharging between recording sessions using magnetic connections that protruded through the elastomer from the PCB base. The circuit was mounted in the center on a thin layer of silicone gel to allow a greater range of bending without skin delamination. The study device included a strain isolator located above each electrode and surrounded the electrodes to shield them from excessive or sudden strain while having exposed elastomer directly over the electrodes free to maintain conformal contact at the skin-electrode interface. The studied device 610 was configured to be placed on the chest area and evaluated under various daily activities, such as standing, walking, running, and sudden arm movements.

Mechanical Properties. The studied device 610 was configured to have all necessary components, and a rechargeable battery has mechanical flexibility and stretchability. Panel 650 shows the studied device 610 capable of being twisted, bent, and stretched beyond the expected deformation of intended application sites on the upper torso.

Data Processing System and Workflow. Diagram 660 shows the data processing workflow for the strain isolated sensor 610 and its corresponding data acquisition system, which included portable smart device 662 for signal monitoring/storage. The measured data from the electrodes (e.g., sensor pad 108, shown as “Nano-membrane Electrodes” 664) and an onboard accelerometer (shown as “6-axis accelerometer” 666) are transmitted via Bluetooth circuity (668) to the user's smartphone or tablet 662 for real-time display or recording of physiological signals. Circuit 690 shows the layout of the studied device (31 mm×21 mm), which includes an antenna, accelerometer, Bluetooth microcontroller, voltage regulator, charging/power management circuit, amplifier, ADC circuit, and electrode input.

The portable smart device 662 was configured with a custom-designed application to display real-time ECG data, 3-axis angular orientation data, and 3-axis acceleration. During study, ECG annotation and long-term health data were calculated at the end of each session.

Performance Validation. FIG. 7 shows the analytical framework for strain-isolation physics and computational modeling. Model 702 shows a section of skin and a single circular electrode that is subjected to a biaxial strain. The shaded portion (exaggerated for emphasis) represents the human skin previously in contact with the electrode before stretching. Model 702 shows the areal change (δA) is directly proportional to the local strain at the electrode. The strain defines the stretch ratios (λ1, λ2), which were calculated from the final dimensions (a, b) and the electrode radius (r). This is applicable for electrodes of any size. Mesh electrodes that can stretch with the skin are also subjected to a disturbance proportional to the strain at each mesh pad. The goal of the modeling is to prevent the temporary changes in contact impedance caused by skin strain and electrode movement or sliding.

Finite Element Analysis. The analysis quantified the stretch ratios and the proportional relationship between the strain and change in the area. Finite element analysis (FEA) was performed, and results were shown in result output 712 with the applied tensile strain (15%) in the vertical direction to mimic a stretched human skin.

Commercial software ABAQUS was used to validate analytical calculations and optimize mechanical performance. The three main components considered were the elastomer substrate, the PCB circuit, and the strain isolation layer. All components were meshed using hexahedral elements: elastomer (C3D8RH), PCB circuit (C3D8), and strain isolation layer (C3D8R), with 787 total elements and 1876 total nodes. The elastomer substrate was modeled as a hyperelastic Neo-Hooke material with coefficients D₁=10.152, C₁₀=4.8 E⁻⁰². The elastic modulus (E) and Poisson's ratio (v) are EPCB=24 GPa, vPCB=0.12, ESIL=1.22 GPa, vSIL=0.43.

The FEA result showed that the bottom electrode, without the integrated SIL, experienced 36% strain, while the top electrode that is shielded by the strain isolator has a calculated strain of 3%. The result also showed that a change in contact area for each electrode is proportional to the strain, meaning that the total area of skin sliding past each tiny electrode pad on the bottom electrode could be over 12 times the change occurring in the top electrode. It is clear from the FEA results that the strain isolator has less change in strain and contract area.

Physical Experiment. The study hypothesized that the strain isolator should be sufficiently rigid to resist in-plane strain while also sufficiently flexible to bend out-of-plane for conformal lamination to the non-flat human skin. A physical experiment was conducted that used a sheet of polypropylene to fabricate a strain isolator. The Young's modulus (E) of that strain isolator E=1.22 GPA using a bending test. Plot 760 shows a Force versus Displacement measurement, and Plot 770 shows a Force versus Displacement measurement for the converted force and Young's modulus.

Adhesion Test. Plot 720 shows the results of an adhesion test from two sample thicknesses versus the bending radius for each trial. The area 722 in the plot is bounded at the top by analytical calculations for the adhesion energy of the elastomer. The result showed that an allowable maximum strain-isolator thickness (of this material) of about 0.3 mm would be capable of maintaining adhesion while being bent around an assumed radius of 15 mm. The result also showed that a minimum thickness that would be capable of reducing strain at the electrode is about 14 μm. Description to determine the strain isolator parameters are discussed in relation to FIG. 3A.

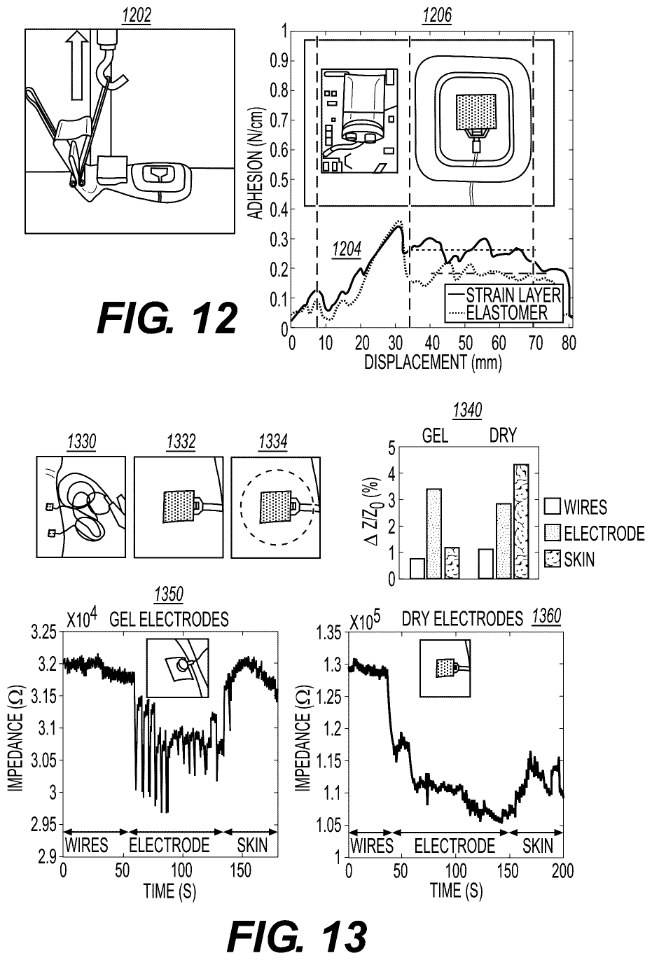

FIG. 12 shows an experimental setup 1202 to measure adhesion strength on a sensor device (with and without the strain isolator) placed on the forearm of a person. Plot 1204 shows adhesion versus displacement measurement for the two tested cases. It was observed that the sensor device with the strain isolator had, on average, an adhesive strength of 0.2614 N/cm (e.g., between 34 mm to 70 mm where the initial rigidity from the circuit components is overcome) as compared to an adhesive strength of 0.1832 N/cm for a sensor device without the strain isolator.

Principal Strain and Von Mises Stress Simulation. FIG. 7 shows additional FEA results 730 for assessments of principal strain and Von Mises stress of the maximum and minimum SIL thicknesses. The results 732 for a strain isolator device (0.3 mm thick) show a 3% strain at the electrode and stress of 4.9 MPa within the SIL. The result 734 for a strain isolator device (14 μm thick) shows an increased strain of 21% at the electrode and internal stress of 58 MPa, which is higher than the yield strength of the material, resulting in partial plastic deformation. The study built the device 610 with a 0.3 mm-thick strain-isolator per this evaluation.

Daily Activity Evaluation. To evaluate the performance of the fabricated strain isolated sensor, the study conducted a series of short and repeatable comparison tests on an indoor course. Each trial (3 minutes long each) consisted of 30-second intervals for a sequence of activities: idle (0 mph), walking (2 mph), fast walking (4 mph), jogging (6 mph), walking (2 mph), and idle (0 mph). The same device was used for eight total trials, four of which occurred before the strain isolator was mounted to a bare elastomer substrate, and four trials were conducted with the strain-isolator integrated into the sensor device. Plot 740 shows representative data. It can be observed that the strain-isolated sensor achieved a clear reduction of motion artifact as compared to a bare elastomer case without the strain isolator structure.

Plot 750 shows the calculated signal-to-noise ratio (SNR) from all trials. It can be observed that the mean SNR reduction from idle to jog was 31.6 to 16.9 dB for the non-strain isolated device as compared to 30.3 to 22.0 dB for the strain isolated device.

Signal Processing and Classification Performance. FIG. 8 shows results from an evaluation of the signal processing and classification performance of the strain-isolated sensor system in a study. The study employed a sensor system that included a pair of electrodes, a 3-axis accelerator, and a 3-axis gyroscope. Flowchart 802 shows the signal processing operations to extract ECG annotation, HR data, RR data, and activity classification. Examples of the acquired signals are shown: HR peak finding (per graph 822), RR peak detection (824), HR and RR moving average (826), and accelerometer data (828) with activity labels (idle, walk, fast walk, and jog) for a three-minute testing routine.

Per flowchart 802, the raw ECG signal was initially filtered using a 0.5-30 Hz bandpass filter to remove low-frequency baseline wander and higher frequency noise, such as chest muscle activities caused by arm motions. A peak-finding algorithm was used to identify local maximum data points, known as the ECG waveform's R-peaks, shown as dots in the HR peak finding graph 822. The HR data were averaged using a 10-second window. Simultaneously, the HR peaks were determined using cubic spline interpolation. The resulting waveform, shown as the dashed red line in 824, can be attributed to cyclic expansion and contraction of the chest during respiration which caused the electrodes to move farther apart from each other and farther from the heart with each inhalation. This change in the R-peak amplitude can be processed with a separate peak-finding algorithm to identify the RR peaks (black triangles in RR peak detection 824), which was averaged using a 30-second window. The HR and RR moving averages (826) were displayed for the entire three-minute testing routine with units of beats-per-minute and respirations-per-minute, respectively.

Separately the accelerometer data 828 were processed to categorize activity levels. The three-axis linear acceleration was used to calculate the total linear acceleration, shown as a black line, using atotal=sqrt (aₓ²+a_{y}²+a_{z}²), which provided a consistent measurement of body motion that is independent of device orientation. A machine-learning algorithm based on the residual convolutional neural network (CNN) classified the user's activities, displayed as idle (832), walk (834), fast walk (836), and jog (838) in the accelerometer data. Total time for each category was used to track daily activity.

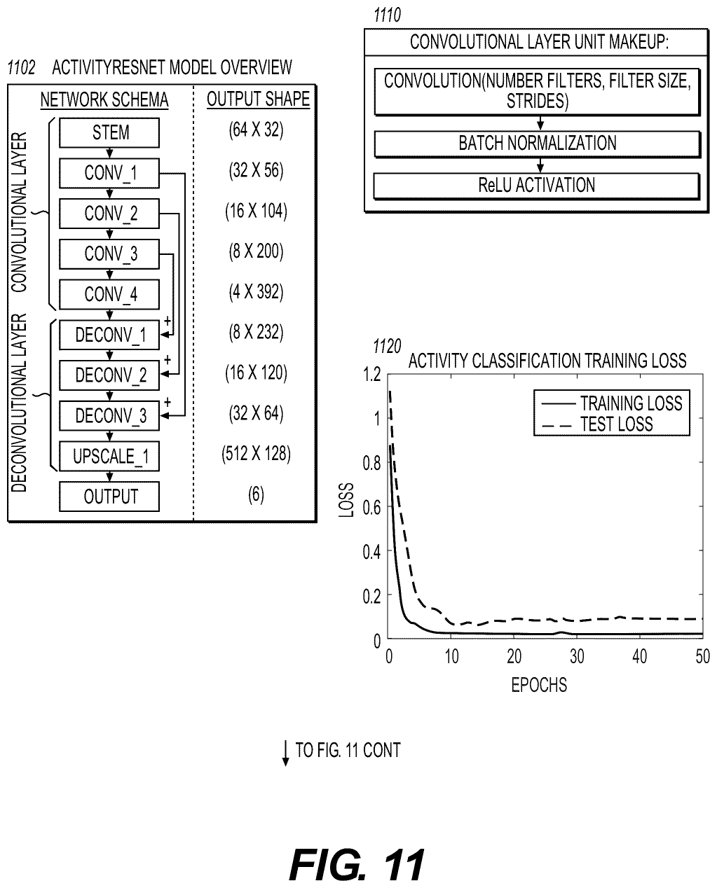

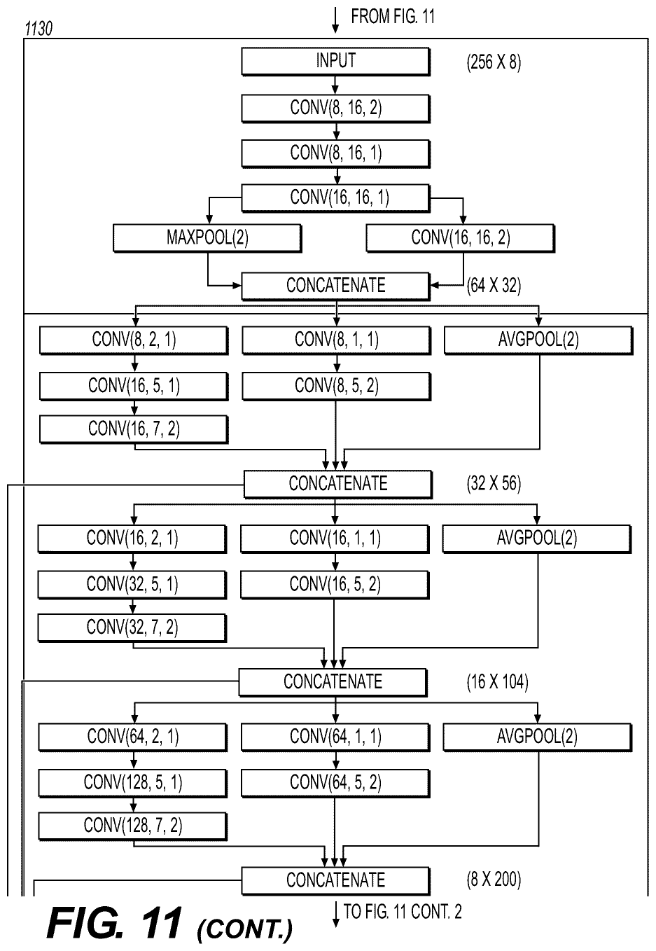

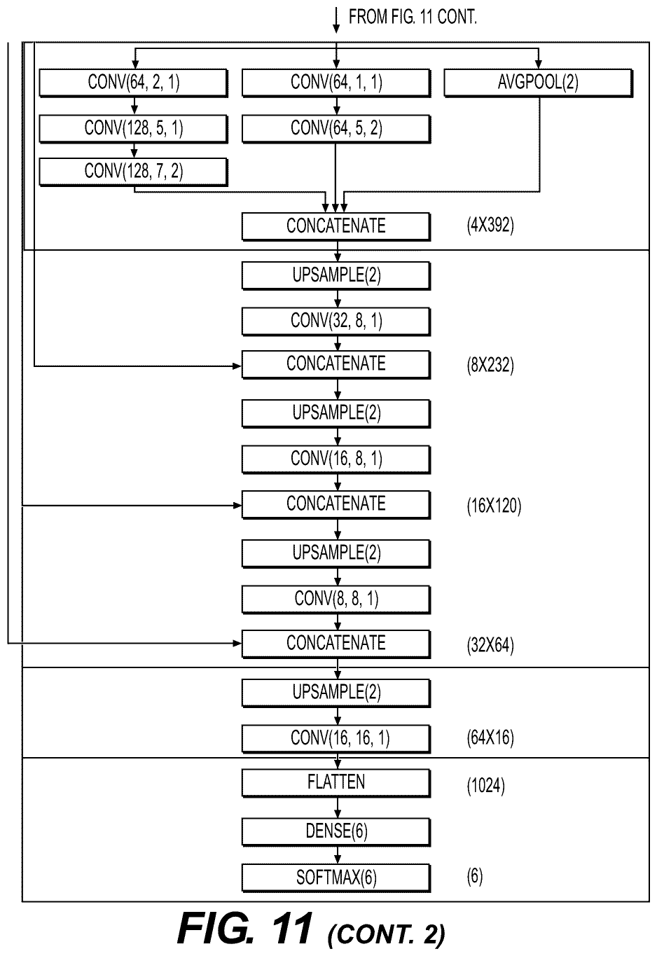

Classification Algorithm. Diagram 840 shows a high-level CNN configuration used for the classification of the 6-axis accelerometer/gyroscope data. Twelve recorded data sets were used to train the model, giving an overall accuracy of 99.3% to recognize the real-time activities of a user, as shown in graph 850. A detailed model with necessary layer components, residual connections, and training test process appears in FIG. 11.

FIG. 11 shows a detailed example of the classification algorithm of FIG. 8. The ActivityResNet model 1102 is shown that includes a set of convolution and deconvolution layers. Diagram 1130 shows a detailed implementation of the ActivityResNet model 1102. Each individual convolutional layer is shown in model 1110. A graph of test loss and training loss 1120 is also shown. The study observed that an embodiment including an all-in-one SIS can successfully measure multiple health-related data by using a computing device (e.g., a user's smartphone). Primary data available in real-time, such as average HR or activity score, can help a patient or athlete evaluate daily health conditions. Beyond that, access to the raw data enables healthcare providers to learn much more about the patient's physiological state through deeper analysis performed after the recording session has ended. The CNN activity classification was trained using data from one participant, giving a reference point that can be used to classify data from any participant guaranteeing a baseline score for each activity. The model can be retrained to fit individual movement patterns or desired fitness goals. Clearly, an advantage of the chest-mounted strain-isolated sensor is that it can be more accurate in ECG, HR, RR, and activity detection than the existing wrist-wearable commercial health monitors.

Comparison Study. The study conducted performance validation of the strain-isolated sensor compared to commercial devices. The study utilized two commercial wireless heart monitors via simultaneous ECG recording on the chest area. FIG. 9 shows the results from the comparison study.

In diagram 902, the strain-isolated device 610 is shown along with a commercial all-in-one device 906 (MAX-ECG Monitor, Maxim Integrated, Inc.) to be placed on the chest. Although both devices were observed to have a similar footprint, the commercial all-in-one device 906 contained a stiffer fabric-backed adhesive gel electrode patch that can be observed to buckle and delaminate from the skin. The strain experienced across the entire length of the patch likely became concentrated at the edge where the circuit is connected, resulting in delamination (908) directly over the electrode. This observation highlights the SIS's advantage of tuned stiffness specifically at the electrode while still allowing the rest of the device to have conformal contact to the skin that can endure body movements.

Plot 910 shows SNR results from four simultaneous tests with mean idle values normalized for comparison. The mean SNR reduction for “idle” and “jog” was 25.7 dB and 12.1 dB, respectively, for the commercial device, as compared to 25.7 dB and 21.6 dB for the strain isolated device. This is a reduction of signal quality by 53% for the commercial device versus 16% for the SIS when compared to their respective baseline idle signals.

Plots 920 shows the raw ECG plots (2-second) comparisons for idle, walk, and jog from one trial. The results show the increased motion artifact on the commercial device as the user's activity level increased, but not for the strain isolated sensor.

Plots 930 show a full exercise session with two devices: normalized ECG waveforms (top), extracted HR and RR (middle), and acceleration data with corresponding activities (bottom).

Plot 940 shows a comparison of the measured ECG data from the strain isolated device and the commercial device when a subject had constant arm movements. The plot shows significant motion artifacts by the commercial device.

A second commercial wireless device with gel electrodes (BioRadio, Great Lakes NeuroTechnologies) was also evaluated. As described by prior articles[23, 30, 36], the BioRadio with hanging wires and gel electrodes showed a significant reduction of SNR and more fluctuation and MA vulnerability.

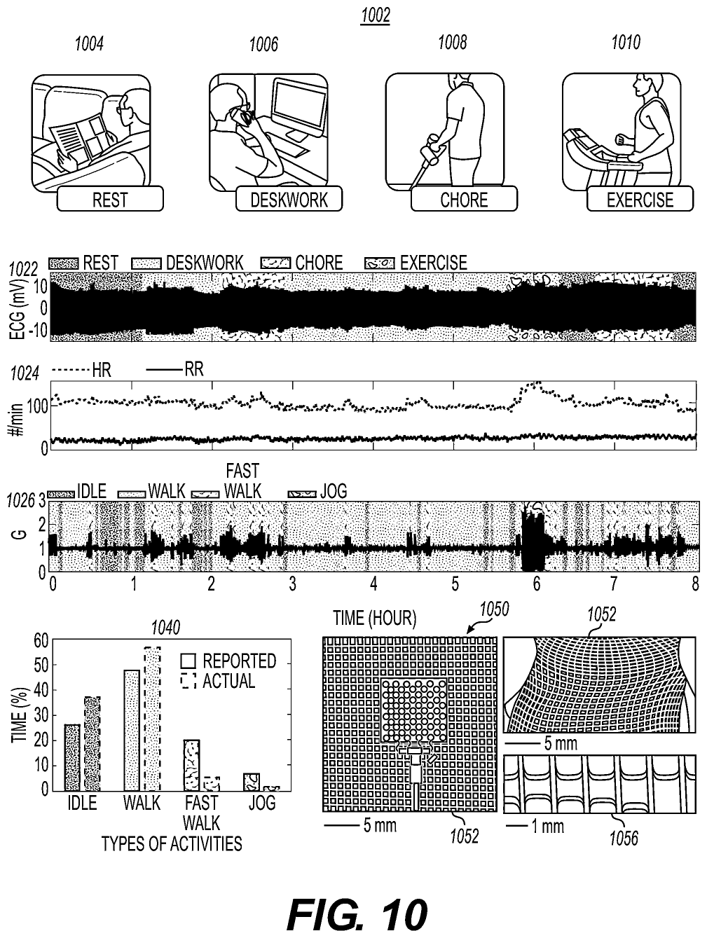

Long-Term Performance Assessment. FIG. 10 shows the results of the study to evaluate the long-term performance of the strain-isolated sensor system, e.g., to measure physiological signals during real-life activities, including rest (1004), deskwork (1006), household chores (1008), and exercises (1010).

Plot 1022 shows a representative set of recorded ECG data of a real-time, continuous recording of physiological data for 8-consecutive hours. Plot 1024 shows the recorded HR and RR data over the same period. Plot 1024 shows the classification output for the trained machine learning for the same period. No motion artifact issue or incident was observed.

Plot 1040 shows a comparison of the percentage of time spent on four types of activities. To facilitate long-term continuous physiological monitoring on the skin, the strain isolated device was configured for continuous skin-electrode contact and low impedance. The device was also configured for maximized air permeation to minimize the effect of excessive sweating during the recording session by employing a breathable, long-term, wearable, perforated substrate. Image 1050 shows the substrate of the studied strain isolated device. The soft elastomeric substrate 1052 includes an array of stretchable breathing holes. Image 1056 shows a cross-section view of the holes.

### Discussion

Cardiovascular diseases affect 48% of the adult population in the United States and continue to be the number one cause of death worldwide [1]. Portable, long-term, continuous monitoring of ECG is urgently needed to detect the onset of various arrhythmias that can happen anytime during daily activities. Many ambulatory ECG devices have been developed to provide smaller form factors than the gold-standard Holter monitor. Collecting high-quality data outside the clinical setting remains challenging due to motion artifacts. An ECG motion artifact (MA) is defined here as the temporary change in measured voltage caused by the movement of the sensor and/or body where the sensor is located. For example, walking creates a downward force on the skin and ECG device with every step, which causes temporary stretching of the skin and relative motion of the skin with the electrode. Together, these two disturbances change the half-cell potential of the skin as well as the contact impedance with the electrode respectively [2]. These temporary changes in the measured voltage can have the same amplitude and frequency as the heart rate[3], making them difficult to distinguish from many physiological signals. Although software algorithms and signal filtering are commonly used to improve signal quality, they are computationally expensive, especially for long-term monitoring, and still only provide an estimate of the actual biosignal [4-7]. Filtering can also be done on any signal as a secondary improvement method but is incapable of improving the raw data. Another solution is to use pressurized tight straps to restrict device movement on the skin[8, 9]. However, this method causes severe discomfort and restriction on the user's activities. If a user loosens the strap pressure, the sensor loses the proper contact with the skin, resulting in signal degradation. Some devices use conductive gels to reduce impedance or strong adhesives to reduce movement, but this often creates skin rash after extended use or even skin breakdown when removed [10-14]. Recent studies have shown possible applications of dry, adhesive-free electrodes that make gentle lamination on the skin [15-17]. However, these still suffer from excessive MA caused by multiple wires, the rigidity of sensors and electronics, and the electrode's movement on the skin. Dry electrodes are especially sensitive to skin strain and vibration induced by body motion while walking, reaching, and performing other daily activities [4, 18-20]. One improvement in dry electrode design was the development of thin-film, open-mesh electrodes capable of stretching with the skin. Previous works have shown some reduction of MA using the mesh electrodes compared to rigid electrodes due to their conformal contact [21-25]. Others have shown the correlation between signal quality and electrode contact area [25-27]. This led us to focus on the mechanisms causing changes to conformal contact and electrode contact area, showing that skin strain at the electrode is the main source of MA for dry electrodes. Although recently reported devices have used soft materials [28-32] or serpentine patterns [33-35], no prior work has shown the capability of a wearable device to reduce MA caused by skin-electrode strain from external sources significantly.

Embodiments of the present disclosure include a fully-integrated, wireless, long-term usable system (SIS) that can physically restrict MA during multi-hour, real-life activities. Embodiments of the present disclosure can include a breathable soft membrane including natural adhesion to offer a skin-friendly, comfortable, continuous recording of multiple biopotentials on the skin. A study is described herein, including the materials, mechanical designs, and soft packaging strategies, along with the details of the strain mechanics at the skin-electrode interface. In addition, the study described strain-isolation design parameters needed for the device to maintain conformal contact to the skin while simultaneously shielding the electrode from excessive skin strain and vibration during patient movement. A set of computational and experimental studies validate the mechanical reliability of the flexible and stretchable device. Embodiments of a signal processing workflow are also disclosed and described and show the monitoring of ECG, heart rate (HR), respiratory rate (RR), and activity classification. In a study described herein, the SIS was simultaneously tested alongside two commercially available wireless devices to show the MA reduction during various physical activities in daily life. In one aspect of a study described herein, the device was worn by multiple participants for over eight hours. During the eight hour period, the participants performed various daily activities ranging from deskwork to exercise. In the studies described herein, an example embodiment of a soft wearable SIS showed outstanding performance of high-quality, continuous, wireless detection of multiple physiological data without MA-based data loss.

This is a continuation of our previous efforts to record high-quality physiological data with a skin-friendly device [15,23,30,36]. While some device designs presented by other groups aim to reduce strain at the electrodes using stiff adhesive patches or fabric backings [8,9], they result in increased stiffness and rigidity of the device, losing the conformal electrode contact to the skin as well as consistent signal quality. On the other hand, the exemplary strain isolated sensors maintain the quality of skin-electrode contact via a soft elastomeric membrane while limiting the excessive strain transmission to the electrode via the strain isolator integration. Thus, the strain isolated sensor can offer continuous, high-quality health monitoring in real-life activities at home or in clinical settings.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include the one particular value and/or the other particular value.

By “comprising” or “containing” or “including” is meant that at least the name compound, element, particle, or method step is present in the composition or article or method but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

As discussed herein, a “subject” may be any applicable human, animal, or other organism, living or dead, or other biological or molecular structure or chemical environment, and may relate to particular components of the subject, for instance, specific tissues or fluids of a subject (e.g., human tissue in a particular area of the body of a living subject), which may be in a particular location of the subject, referred to herein as an “area of interest” or a “region of interest.”

It should be appreciated that, as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to humans (e.g., rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications and publications as listed below and throughout this document are hereby incorporated by reference in their entirety herein.
[1] Benjamin, E.J. et al. Heart Disease and Stroke Statistics-2019 Update: A Report From the American Heart Association. Circulation 139, e56-e528 (2019).
[2] Zhang, Z. et al. Adaptive motion artefact reduction in respiration and ECG signals for wearable healthcare monitoring systems. Medical and Biological Engineering and Computing 52, 1019-1030 (2014).
[3] Kirst, M., Glauner, B. & Ottenbacher, J. Using DWT for ECG motion artifact reduction with noise-correlating signals. Proceedings of the Annual International Conference of the IEEE Engineering in Medicine and Biology Society, EMBS, 4804-4807 (2011).
[4] Liu, Y. & Pecht, M. G. Reduction of Skin Stretch Induced Motion Artifacts in Electrocardiogram Monitoring Using Adaptive Filtering. Proceedings of the 28th IEEE EMBS Annual International Conference (2006).
[5] Kalra, A. & Lowe, A. Development and validation of Motion Artefact Rejection System (MARS) for electrocardiogramg novel skin-stretch estimation approach. Sensors and Actuators, A: Physical 301, 111726-111726 (2020).
[6] Zhang, H. & Zhao, J. Motion artefact suppression method for wearable ECGs. Feature Engineering and Computational Intelligence in ECG Monitoring, 73-88 (2020).
[7] Lee, S.C. & Kim, S.M. Motion artifact reduction algorithm in wearable healthcare system. Journal of Medical Devices, Transactions of the ASME 10 (2016).
[8] Porr, B. & Howell, L. R-peak detector stress test with a new noisy ECG database reveals significant performance differences amongst popular detectors. bioRxiv, 1-27, doi: 10.1101/722397 (2019).
[9] MAX-ECG-MONITOR User Guide. 15 (2018).
[10] Barrett, P.M. et al. Comparison of 24-hour Holter monitoring with 14-day novel adhesive patch electrocardiographic monitoring. American Journal of Medicine 127, 95.e11-95.e17 (2014).
[11] Rho, R., Vossler, M., Blancher, S. & Poole, J.E. Comparison of 2 ambulatory patch ECG monitors: The benefit of the P-wave and signal clarity. American Heart Journal 203, 109-117 (2018).
[12] Nault, I. et al. Validation of a novel single lead ambulatory ECG monitor—Cardiostat—Compared to a standard ECG Holter monitoring. J Electrocardiol 53, 57-63 (2019).
[13] Upadhyayula, S. & Kasliwal, R. Wellysis S-PAtch Cardio versus Conventional Holter Ambulatory Electrocardiographic monitoring (The PACER Trial): Preliminary Results. Journal of Clinical and Preventive Cardiology 8, 173-173 (2019).
[14] Walsh, J.A., Topol, E.J. & Steinhubl, S.R. Novel wireless devices for cardiac monitoring. Circulation 130, 573-581 (2014).
[15] Herbert, R., Kim, J.-H., Kim, Y.S., Lee, H.M. & Yeo, W.-H. Soft material-enabled, flexible hybrid electronics for medicine, healthcare, and human-machine interfaces. Materials 11, 187 (2018).
[16] Lim, H.R. et al. Advanced soft materials, sensor integrations, and applications of wearable flexible hybrid electronics in healthcare, energy, and environment. Advanced Materials 32, 1901924 (2020).
[17] Fu, Y., Zhao, J., Dong, Y. & Wang, X. Dry Electrodes for Human Bioelectrical Signal Monitoring. Sensors (Basel) 20 (2020).
[18] Ottenbacher, J. et al. Reliable motion artifact detection for ECG monitoring systems with dry electrodes. Proceedings of the 30th Annual International Conference of the IEEE Engineering in Medicine and Biology Society, EMBS′08—“Personalized Healthcare through Technology”, 1695-1698 (2008).
[19] Chi, Y.M., Jung, T.P. & Cauwenberghs, G. Dry-contact and noncontact biopotential electrodes: Methodological review. IEEE Reviews in Biomedical Engineering 3, 106-119 (2010).
[20] Heikenfeld, J. et al. Wearable sensors: Modalities, challenges, and prospects. Lab on a Chip 18, 217-248, doi: 10.1039/c7lc00914c (2018).
[21] Jeong, J.W. et al. Materials and optimized designs for human-machine interfaces via epidermal electronics. Advanced Materials 25, 6839-6846 (2013).
[22] Kwon, Y.-T. et al. All-printed nanomembrane wireless bioelectronics using a biocompatible solderable graphene for multimodal human-machine interfaces. Nature communications 11, 1-11 (2020).
[23] Kim, H. et al. Fully Integrated, Stretchable, Wireless Skin-Conformal Bioelectronics for Continuous Stress Monitoring in Daily Life. Advanced Science 7, 2000810 (2020).
[24] Tian, L. et al. Large-area MRI-compatible epidermal electronic interfaces for prosthetic control and cognitive monitoring. Nature Biomedical Engineering 3, 194-205 (2019).
[25] Huigen, E., Peper, A. & Grimbergen, C. A. Investigation into the origin of the noise of surface electrodes. Med. Biol. Eng. Comput. 40, 332-338 (2002).
[26] An, X. & Stylios, G.K. A hybrid textile electrode for electrocardiogram (ECG) measurement and motion tracking. Materials 11, 1887 (2018).
[27] Tasneem, N.T., Pullano, S.A., Critello, C.D., Fiorillo, A.S. & Mahbub, I. A low-power on-chip ecg monitoring system based on mwcnt/pdms dry electrodes. IEEE Sensors Journal 20, 12799-12806 (2020).
[28] Chung, H.U. et al. Skin-interfaced biosensors for advanced wireless physiological monitoring in neonatal and pediatric intensive-care units. Nature Medicine 26, 418-429 (2020).
[29] Chung, H.U. et al. Binodal, wireless epidermal electronic systems with in-sensor analytics for neonatal intensive care. Science 363, 0-13 (2019).
[30] Kim, Y.-S. et al. All-in-One, Wireless, Stretchable Hybrid Electronics for Smart, Connected, and Ambulatory Physiological Monitoring. Advanced Science 6 (2019).
[31] Lin, R., Li, Y., Mao, X., Zhou, W. & Liu, R. Hybrid 3D Printing All-in-One Heterogenous Rigidity Assemblies for Soft Electronics. Advanced Materials Technologies 4, 1-8 (2019).
[32] Liu, Y. et al. Intraoperative monitoring of neuromuscular function with soft, skin-mounted wireless devices. npj Digital Medicine 1 (2018).
[33] Zulqarnain, M. et al. A flexible ECG patch compatible with NFC RF communication. npj Flexible Electronics 4, 1-9 (2020).
[34] Dong, W., Cheng, X., Xiong, T. & Wang, X. Stretchable bio-potential electrode with self-similar serpentine structure for continuous, long-term, stable ECG recordings. Biomedical Microdevices 21 (2019).
[35] Li, Y. et al. A Stretchable-Hybrid Low-Power Monolithic ECG Patch with Microfluidic Liquid-Metal Interconnects and Stretchable Carbon-Black Nanocomposite Electrodes for Wearable Heart Monitoring. Advanced Electronic Materials 5, 1-12 (2019).
[36] Kim, Y.-S. et al. Wireless, skin-like membrane electronics with multifunctional ergonomic sensors for enhanced pediatric care. IEEE Transactions on Biomedical Engineering 67, 2159-2165 (2019).
[37] Goodno, B. J. & Gere, J. M. Mechanics of materials. Ninth Edition edn, (Cengage Learning, 2018).
[38] Tompkins, W. J. & Pan, J. A Real-Time QRS Detection Algorithm. IEEE TRANSACTIONS ON BIOMEDICAL ENGINEERING 32 (1985).
[39] Fung, E. et al. Electrocardiographic patch devices and contemporary wireless cardiac monitoring. Frontiers in Physiology 6 (2015).
[40] Berwal, D., Vandana, C.R., Dewan, S., Jiji, C.V. & Baghini, M.S. Motion Artifact Removal in Ambulatory ECG Signal for Heart Rate Variability Analysis. IEEE Sensors Journal 19, 12432-12442 (2019).

1. A system comprising:
a flexible substrate comprising two or more low-modulus layers, including a top low-modulus layer and a bottom low-modulus layer, the flexible substrate having a first side on the top low-modulus layer and a second side on the bottom low-modulus layer, wherein the second side is configured as a breathable soft membrane configured to directly contact and adhere with a skin region of a person;
one or more pads fixably attached to the second side of the flexible substrate, wherein the one or more pads include a first pad that attaches to the second side over a first area, wherein the one or more pads each has a side that is exposed to directly contact a portion of the skin region; and
one or more strain-isolating structures fixably attached to the top low-modulus layer, including a first strain-isolating structure, wherein the first strain-isolating structure is attached to the top low-modulus layer at an area corresponding to the first area of the first pad and is shaped to have an outer dimension that forms a perimeter around the first pad.

2. The system of claim 1, wherein the first strain-isolating structure has an inner dimension that extends beyond the first area of the first pad.

3. The system of claim 1, wherein the first pad has a first shape and the first strain-isolating structure has a second shape, wherein the first shape of the first pad is the same as the second shape of the first strain-isolating structure.

4. The system of claim 1, wherein the first pad has a first shape and the first strain-isolating structure has a second shape, wherein the first shape of the first pad is different from the second shape of the first strain-isolating structure.

5. The system of claim 1, wherein the first pad has a shape selected from the group consisting of a square area, a rectangular area, a circular area, or an oval area.

6. (canceled)

7. The system of claim 1, wherein the top low-modulus layer comprises a thin low-modulus silicone elastomer material having an average thickness less than 1000 um.

8. The system of claim 1, wherein the bottom low-modulus layer comprises a low modulus silicone gel material having a modulus value less than 20 kPa.

9. The system of claim 1, wherein the top low-modulus layer comprises a first material having a first modulus value and the bottom low-modulus layer comprises a second material having a second modulus value, wherein the first modulus value of the top low-modulus layer is at least 2 times that of the second modulus value of the bottom low-modulus layer.

10. The system of claim 1, wherein the one or more pads form an array.

11. The system of claim 1, wherein the one or more pads are configured as an open mesh stretchable electrode.

12. (canceled)

13. The system of claim 1, wherein the system is configured as a smartwatch configured for at least one of optical measurement, impedance measurement, capacitance measurement, or voltage potential measurement through the stretchable pads.

14. The system of claim 1, further comprising:
a second stretchable pad that attaches to the second side over a second area, and
a second strain-isolating structure attached to the top low-modulus layer at a third area corresponding to the second area of the second stretchable pad and is shaped to have an outer dimension that forms a perimeter around the second stretchable pad.

15. The system of claim 14, wherein one or more pads, including the first pad, form a pair of stretchable contacts that is fixably attached to the second side of the flexible substrate over a second area,
the system further comprising:
an active integrated sensor component that couples to the pair of stretchable contacts.

16. (canceled)

17. The system of claim 1, wherein the system is configured as an electrocardiographic probe.

18. The system of claim 17, wherein the electrocardiographic probe includes a pair of the flexible pads, a multi-axis accelerator, and a multi-axis gyroscope.

19. The system of claim 17, wherein the electrocardiographic probe further includes an optical sensor, a photodiode, a capacitance, and/or a temperature sensor.

20. The system of claim 1, further comprising:
an active integrated chip assembly mounted on the flexible substrate, wherein the active integrated chip assembly comprises at least one or more active integrated circuits, including a transimpedance amplifier circuit and a digital-to-analog convertor.

21. The system of claim 20, further comprising:
an encapsulation layer that encapsulates the active integrated chip assembly.

22. The system of claim 20, wherein the active integrated chip circuit further includes a local processing unit or controller configured to provide measured signal data to a device processing unit or controller.

23. The system of claim 21, wherein the device processing unit or controller is configured to employ at least one of electrocardiogram signals, heart rate signals, respiration rate signals, or a combination thereof acquired through an electrode or sensor associated with the one or more pads.
